(19) United States
(12) Patent Application Publication
(10) Pub. No.: US 2023/0255525 A1
(43) Pub. Date: Aug. 17, 2023

(54) ANALYSIS METHOD AND NEAR INFRARED SPECTROSCOPY SENSOR

(71) Applicant: HONDA MOTOR CO., LTD., Tokyo (JP)

(72) Inventors: Daisuke KURASHINA, Saitama (JP); Ryuichi KUBOTA, Saitama (JP); Teruyuki USHIJIMA, Saitama (JP)

(21) Appl. No.: 18/154,865

(22) Filed: Jan. 16, 2023

(30) Foreign Application Priority Data
Feb. 14, 2022 (JP) 2022-020321

(51) Int. Cl. A61B 5/1455; A61B 5/145; A61B 5/00

(52) U.S. Cl. CPC A61B 5/14552; A61B 5/14546; A61B 5/7278; A61B 2562/0238; A61B 2562/0285; A61B 5/01

(57) ABSTRACT

An analysis method is provided which includes providing nanodiamond in a vicinity of a sample; and measuring a content of a compound having a near infrared absorption peak amplified by the nanodiamond in the sample using near infrared spectroscopy. Also, a near infrared spectroscopy sensor is provided which includes a near infrared spectroscopy sensor main body including a layer containing nanodiamond on a surface that receives near infrared rays of the near infrared spectroscopy sensor main body.

This application is based on and claims the benefit of priority from Japanese Patent Application No. 2022-020321, filed on 14 Feb. 2022, the content of which is incorporated herein by reference.

### BACKGROUND OF THE INVENTION

### Field of the Invention

The present invention relates to an analysis method and a near infrared spectroscopy sensor.

### Related Art

Conventionally, various methods have been used as methods for quantitatively analyzing components of blood gas noninvasively. For example, a pulse oximeter is used as an apparatus for nondestructively measuring percutaneous arterial oxygen saturation (SpO₂) (refer to Japanese Unexamined Patent Application, Publication No. 2015-177955). This makes it possible to evaluate whether oxygen is being normally supplied to blood.

On the other hand, since nitrogen monoxide produced from vascular endothelial cells has physiological activities such as vasodilatory action, platelet aggregation inhibiting action, vascular smooth muscle proliferation inhibiting action, and the like, attention has been paid from the viewpoint of hypertension, arteriosclerosis, thrombosis, and the like.
Patent Document 1: Japanese Unexamined Patent Application, Publication No. 2015-177955

### SUMMARY OF THE INVENTION

For this reason, it is desirable to analyze the concentration of nitrogen monoxide in blood and in blood gas with high accuracy noninvasively.

It is an object of the present invention to provide an analysis method and a near infrared spectroscopy sensor capable of accurately analyzing concentrations of nitrogen monoxide in blood and in a blood gas noninvasively.

One embodiment of the present invention is directed to an analysis method including: providing nanodiamond in a vicinity of a sample; and measuring a content of a compound having a near infrared absorption peak amplified by the nanodiamond in the sample using near infrared spectroscopy.

The compound may be nitrogen oxide.

The above analysis method may further include: providing the nanodiamond at a predetermined portion of a subject; and measuring a concentration of nitrogen monoxide in blood flowing through a blood vessel existing in the vicinity of a predetermined portion of the subject using the near infrared spectroscopy.

The above analysis method may further include: converting a wavelength of a near infrared absorption peak of the nitrogen monoxide into a deep body temperature of the subject.

Another embodiment of the present invention is directed to a near infrared spectroscopy sensor comprising a near infrared spectroscopy sensor main body including a layer containing nanodiamond on a surface that receives near infrared rays of the near infrared spectroscopy sensor main body.

According to an embodiment of the present invention, it is possible to provide an analysis method and a near infrared spectroscopy sensor capable of accurately analyzing concentrations of nitrogen monoxide in blood and in a blood gas noninvasively.

### BRIEF DESCRIPTION OF THE DRAWINGS

FIG. 1 is a diagram showing an example of an analysis method according to an embodiment of the present invention;

FIG. 2 is a diagram showing an example of a near infrared absorption spectrum obtained by the analysis method of FIG. 1;

FIG. 3 is a diagram illustrating a shift of a wavelength of a near infrared absorption peak derived from nitrogen monoxide with a change in a deep body temperature of a subject; and

FIG. 4 is a side view showing an example of the near infrared spectroscopy sensor of the present embodiment.

### DETAILED DESCRIPTION OF THE INVENTION

Hereinafter, an embodiment of the present invention will be described with reference to the accompanying drawings.

An analysis method according to an embodiment of the present invention includes a step of providing nanodiamond in the vicinity of a sample, and a step of measuring a content of a compound having a near infrared absorption peak amplified by the nanodiamond in the sample using near infrared spectroscopy.

The compound having a near infrared absorption peak amplified by the nanodiamond is not particularly limited, and examples thereof include nitrogen oxide and ammonia. Examples of the nitrogen oxide include nitrogen monoxide and nitrogen dioxide.

Since the nitrogen oxide has a very small near infrared absorption peak, it is generally difficult to analyze the nitrogen oxide using near infrared spectroscopy. However, when the nanodiamond is provided in the vicinity of the sample, the near infrared absorption peak of the nitrogen oxide is amplified by the interaction of the nanodiamond with the pores. The nanodiamond has a high transmittance for near infrared rays. Therefore, the content of the nitrogen oxide in the sample can be accurately analyzed in a non-destructive manner.

In the nanodiamond, an amorphous layer made of sp² carbon is formed on the surface of a core portion made of sp³ carbon, and an oxygen-containing group exists on the surface. Examples of the oxygen-containing group include a hydroxyl group, a carboxyl group, a carbonyl group, and an oxy group.

Although the particle size of the nanodiamond is not particularly limited, it is, for example, 3 nm or more and 8 nm or less. The nanodiamond may be aggregated.

The method for producing the nanodiamond is not particularly limited, and examples thereof include chemical vapor deposition (CVD).

The method of providing the nanodiamond in the vicinity of the sample is not particularly limited, and examples thereof include a method of applying a coating solution containing the nanodiamond and a method of attaching a film containing the nanodiamond.

When the content of a compound having a near infrared absorption peak amplified by the nanodiamond in the sample is measured by near infrared spectroscopy, a well-known near infrared spectrometer, a near infrared spectroscopy sensor, or the like can be used.

By using the analysis method of the present embodiment, the concentration of nitrogen monoxide in the blood and the concentration of nitrogen monoxide in the blood gas can be accurately analyzed noninvasively. In this case, the analysis method of the present embodiment includes a step of providing the nanodiamond at a predetermined portion of a subject, and a step of measuring the concentration of nitrogen monoxide in blood flowing through a blood vessel existing in the vicinity of a predetermined portion of the subject using near infrared spectroscopy.

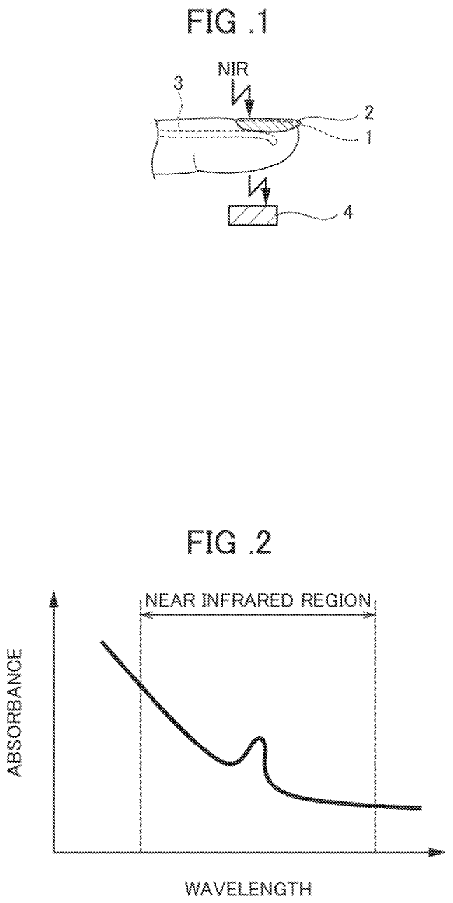

FIG. 1 shows a method of measuring the concentration of nitrogen monoxide in blood and blood gas as an example of the analysis method of the present embodiment.

First, a coating solution 2 containing nanodiamond is applied onto a nail 1 of a subject, and then the coating solution is dried. Next, the nail 1 coated with the coating solution 2 is irradiated with near infrared rays (NIR), and the near infrared spectroscopy sensor 4 receives the near infrared rays (NIR) transmitted through the blood vessel 3 existing in the vicinity of the nail 1. At this time, the near infrared spectroscopy sensor 4 detects the concentration of nitrogen monoxide in the blood and the blood gas based on the area of the near infrared absorption peak derived from nitrogen monoxide contained in the blood flowing through the blood vessel 3 in the near infrared absorption spectrum (refer to FIG. 2). At this time, it is preferable to create in advance a calibration curve indicating the relationship between the area of the near infrared absorption peak derived from nitrogen monoxide and the concentration of nitrogen monoxide in the blood and in the blood gas.

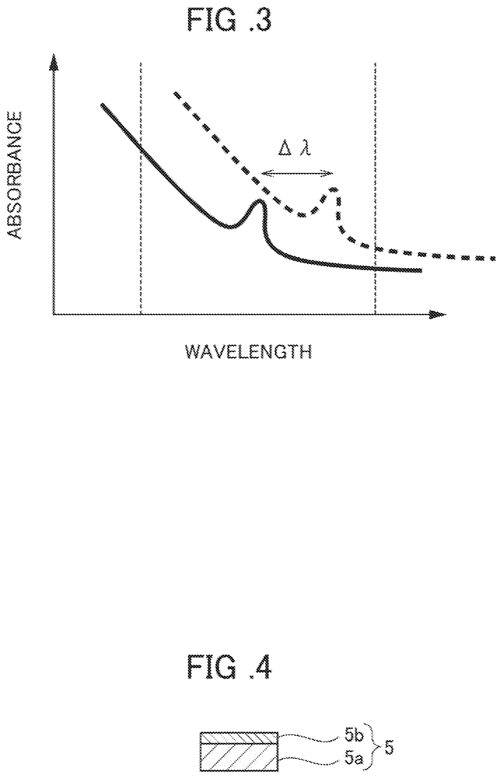

Furthermore, since the wavelength (λ) of the near infrared absorption peak derived from nitrogen monoxide is shifted by (Δλ) with the change (ΔT) in the deep body temperature (T) of the subject (refer to FIG. 3), the wavelength (λ) of the near infrared absorption peak derived from nitrogen monoxide can be converted into the deep body temperature (T) of the subject. More specifically, when the deep body temperature (T) of the subject decreases, the wavelength (λ) of the near infrared absorption peak derived from nitrogen monoxide increases. At this time, it is preferable to create in advance a calibration curve indicating the relationship between the deep body temperature (T) of the subject and the wavelength (λ) of the near infrared absorption peak derived from nitrogen monoxide.

The portion of the subject to which the coating solution 2 containing nanodiamond is applied is not limited to the nail 1 as long as it can detect the concentration of nitrogen monoxide in blood flowing through a blood vessel existing in the vicinity.

Instead of applying the coating solution 2 containing nanodiamond, a film containing nanodiamond may be attached.

Furthermore, instead of applying the coating solution 2 containing nanodiamond onto the nail 1 of the subject, a near infrared spectroscopy sensor 5 (refer to FIG. 4) in which a layer 5b containing nanodiamond is formed on the surface of the near infrared spectroscopy sensor body 5a on the side receiving near infrared rays (NIR) may be used. In this case, the concentration of nitrogen monoxide in blood flowing through a blood vessel existing in the vicinity of the layer 5b containing nanodiamond can be measured by bringing the layer 5b containing nanodiamond of the near infrared spectroscopy sensor 5 into contact with a predetermined portion of the subject.

In addition, the near infrared spectroscopy sensor 5 can be applied to, for example, an exhaust gas sensor in addition to measuring the concentration of nitrogen monoxide in blood and in blood gas. In this case, the concentration of NOₓ in the exhaust gas existing in the vicinity of the layer 5b containing nanodiamond can be measured based on the area of the near infrared absorption peak such as nitrogen oxide (NOₓ), ammonia, or hydrocarbon.

Although an embodiment of the present invention has been described above, the present invention is not limited to the above-described embodiment, and the above-described embodiment may be appropriately modified within the scope of the present invention.

### EXPLANATION OF REFERENCE NUMERALS

1 nail
2 coating solution containing nanodiamond
3 blood vessel
4, 5 near infrared spectroscopy sensor
5a near infrared spectroscopy sensor body
5b layer containing nanodiamond

What is claimed is:

1. An analysis method comprising:
providing nanodiamond in a vicinity of a sample; and
measuring a content of a compound having a near infrared absorption peak amplified by the nanodiamond in the sample using near infrared spectroscopy.

2. The analysis method according to claim 1, wherein the compound is nitrogen oxide.

3. The analysis method according to claim 2, further comprising:
providing the nanodiamond at a predetermined portion of a subject; and
measuring a concentration of nitrogen monoxide in blood flowing through a blood vessel existing in a vicinity of a predetermined portion of the subject using the near infrared spectroscopy.

4. The analysis method according to claim 3, further comprising converting a wavelength of a near infrared absorption peak of the nitrogen monoxide into a deep body temperature of the subject.

5. A near infrared spectroscopy sensor comprising a near infrared spectroscopy sensor main body including a layer containing nanodiamond on a surface that receives near infrared rays of the near infrared spectroscopy sensor main body.
